SEQUENCE LISTING (4 sequences)

SEQ ID NO: 1 - DNA, 33 bp, Artificial
  primer 1
ggaagatctatttgccttcgcacatcaacctgg

SEQ ID NO: 2 - DNA, 30 bp, Artificial
  primer 2
cggggtaccttgtaaatattttaacccgcc

SEQ ID NO: 3 - DNA, 56 bp, Artificial
  primer 3
ggaagatctaaggagaccttaaatgagcgacacaacgatcctgcaaaacagtaccc

SEQ ID NO: 4 - DNA, 39 bp, Artificial
  primer 4
cggggtacctcgtagaggtttactggcgcttatccagcg

(19) United States
(12) Patent Application Publication
(10) Pub. No.: US 2009/0093029 A1
(43) Pub. Date: Apr. 9, 2009

(54) METHOD FOR PRODUCING L-AMINO ACID

(76) Inventors: Yoshihiro Usuda, Kawasaki-shi (JP); Kazuhiko Matsui, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 12/202,484

(22) Filed: Sep. 2, 2008

Related U.S. Application Data
(63) Continuation of application No. PCT/JP2007/053803, filed on Feb. 28, 2007.

(30) Foreign Application Priority Data
Mar. 3, 2006 (JP) 2006-057528

(51) Int. Cl. C12P 13/04; C12P 13/08; C12P 13/22

(52) U.S. Cl. 435/108; 435/106; 435/115

(57) ABSTRACT

An L-amino acid is produced by culturing an Enterobacteriaceae which is able to produce an L-amino acid in a medium containing glycerol, especially crude glycerol, as the carbon source to produce and accumulate the L-amino acid in the culture, and collecting the L-amino acid from the culture.

This application is a continuation under 35 U.S.C. §120 to PCT Patent Application No. PCT/JP2007/053803, filed on Feb. 28, 2007, which claims priority under 35 U.S.C. § 19 to Japanese Patent Application No. 2006-057528, filed Mar. 3, 2006, both of which are incorporated by reference. The Sequence Listing filed electronically herewith is also hereby incorporated by reference in its entirety (File Name: US-370_Seq_List_Copy_{—}1; File Size: 1 KB; Date Created: Sep. 2, 2008)

### BACKGROUND OF THE INVENTION

1. Technical Field

The present invention relates to a method for producing an L-amino acid using a microorganism. L-amino acids are useful in various fields, including for use in seasonings, as food additives, feed additives, and as chemicals and drugs.

2. Background Art

L-amino acids such as L-threonine and L-lysine are industrially produced by fermentation using amino acid-producing bacteria such as Escherichia. Amino acid-producing bacteria include strains isolated from nature, artificial mutants of those bacterial strains, and recombinants of those bacterial strains in which L-amino acid biosynthetic enzymes are enhanced by genetic recombination, or the like. Examples of methods for producing L-threonine include, for example, the methods described in Japanese Patent Laid-open (JP-A, Kokai) No. 5-304969, International Patent Publication WO98/04715, Japanese Patent Laid-open No. 05-227977, and U.S. Patent Published Application No. 2002/0110876. Examples of the methods for producing L-lysine include, for example, the methods described in Japanese Patent Laid-open No. 10-165180, Japanese Patent Laid-open No. 11-192088, Japanese Patent Laid-open No. 2000-253879, and Japanese Patent Laid-open No. 2001-057896.

In the industrial production of L-amino acids by fermentation, saccharides, for example, glucose, fructose, sucrose, blackstrap molasses, starch hydrolysate, and so forth, are typically used as the carbon source.

### SUMMARY OF THE INVENTION

The present invention provides a method for producing an L-amino acid at a low cost by using a raw material not previously used in conventional methods for producing L-amino acids by fermentation using microorganisms, which mainly utilize saccharides as the carbon sources during fermentation.

It is an aspect of the present invention to describe a method of culturing a bacterium belonging to the family Enterobacteriaceae which is able to produce an L-amino acid in a medium containing glycerol as the carbon source, and as a result, an equivalent or higher amount of L-amino acids are produced as compared to when saccharides are used as the carbon source. Furthermore, it is another aspect to provide a crude glycerol of low purity, which is produced as a by-product during the production of biodiesel fuel, which is industrially produced worldwide. This crude glycerol demonstrated a higher growth promoting effect, as compared to pure glycerol.

It is an aspect of the present invention to provide a method for producing an L-amino acid comprising culturing an Enterobacteriaceae which is able to produce an L-amino acid when cultured in a medium containing glycerol as the carbon source, and collecting the L-amino acid from the culture medium.

It is a further aspect of the present invention to provide the method as described above, wherein the concentration of glycerol in the medium at the start of the culture is 1 to 30% w/v.

It is a further aspect of the present invention to provide the method as described above, wherein crude glycerol is added to the medium.

It is a further aspect of the present invention to provide the method as described above, wherein the crude glycerol is produced in biodiesel fuel production.

It is a further aspect of the present invention to provide the method as described above, wherein the use of the crude glycerol as the carbon source in the medium results in production of more L-amino acid than when the reagent glycerol is used as the carbon source in the same culture method.

It is a further aspect of the present invention to provide the method as described above, wherein the bacterium belongs to the genus Escherichia.

It is a further aspect of the present invention to provide the method as described above, wherein the bacterium belongs to the genus Pantoea.

It is a further aspect of the present invention to provide the method as described above, wherein the bacterium is Escherichia coli.

It is a further aspect of the present invention to provide the method as described above, wherein the L-amino acid is selected from the group consisting of L-threonine or L-lysine, L-glutamic acid, L-lysine, and L-tryprophan.

It is a further aspect of the present invention to provide the method as described above, wherein the L-amino acid is L-threonine, and the activity of an enzyme selected from the group consisting of aspartokinase I, homoserine kinase, aspartate aminotransferase, threonine synthase which are encoded by the thr operon, aspartate semialdehyde dehydrogenase, and combinations thereof is increased in the bacterium.

It is a further aspect of the present invention to provide the method as described above, wherein the L-amino acid is L-lysine, and activity of an enzyme selected from the group consisting of dihydrodipicolinate reductase, diaminopimelate decarboxylase, diaminopimelate dehydrogenase, phosphoenolpyruvate carboxylase, aspartate aminotransferase, diaminopimelate epimerase, aspartate semialdehyde dehydrogenase, tetrahydrodipicolinate succinylase, succinyl diaminopimelate deacylase, and combinations thereof is increased, and/or activity of lysine decarboxylase is attenuated, in the bacterium.

It is a further aspect of the present invention to provide the method as described above, wherein the L-amino acid is L-glutamic acid, and activity of an enzyme selected from the group consisting of glutamate dehydrogenase, citrate synthase, phosphoenolpyruvate carboxylase, methyl citrate synthase, and combinations thereof is increased, and/or activity of α-ketoglutarate dehydrogenase is attenuated, in the bacterium.

It is a further aspect of the present invention to provide the method as described above, wherein the L-amino acid is L-tryptophan, and activity of an enzyme selected from the group consisting of phosphoglycerate dehydrogenase, 3-deoxy-D-arabinoheptulonate-7-phosphate synthase, 3-dehydroquinate synthase, shikimate dehydratase, shikimate kinase, 5-enolpyruvate shikimate 3-phosphate synthase, chorismate synthase, prephenate dehydratase, chorismate mutase, and combinations thereof is increased in the bacterium.

### DESCRIPTION OF THE PREFERRED EMBODIMENTS

Hereinafter, the present invention will be explained in detail.

<1> Glycerol

“Glycerol” refers to a substance having the nomenclature name of propane-1,2,3-triol. Crude glycerol refers to industrially produced glycerol, which will contain impurities. Crude glycerol is industrially produced by hydrolyzing fats or oils with water at a high temperature and under high pressure, or during biodiesel fuel production via the esterification reaction. “Biodiesel fuel” refers to the aliphatic acid methyl esters produced from fats or oils, and the methanol produced by transesterification. Crude glycerol is produced as a by-product of this reaction (refer to Fukuda, H., Kondo, A., and Noda, H., 2001, J. Biosci. Bioeng., 92, 405-416). In the biodiesel fuel production process, the alkaline catalyst method is typically used for the transesterification, and acids are added for neutralization. As a result, crude glycerol containing water and impurities is produced, and typically is about 70 to 95% pure by weight. Crude glycerol produced in the biodiesel fuel production contains, in addition to water, residual methanol, alkali salts such as NaOH which acts as a catalyst, and an acid, such as K₂SO₄, which acts to neutralize the alkali. Although it depends on the manufacturer and the production method, the content of such salts and methanol can be several percent. The crude glycerol preferably contains ions, which are generated from the alkali and the neutralizing acid, such as sodium ions, potassium ions, chloride ions, and sulfate ions, which may be present in an amount of from 2 to 7%, preferably 3 to 6%, more preferably 4 to 5.8%, based on the weight of the crude glycerol. Although methanol may not be present, it is preferably present in an amount of 0.01% or less.

The crude glycerol may further contain trace amounts of metals, organic acids, phosphorus, aliphatic acids, and so forth. Examples of the organic acids which may be present include formic acid, acetic acid, and so forth, and although such acids may not be present, they are preferably present in an amount of 0.01% or less. The metals which may be present in the crude glycerol include trace metals which are required for growth of the chosen microorganisms, such as magnesium, iron, calcium, manganese, copper, zinc, and so forth. Magnesium, iron, and calcium may be present in an amount of from 0.00001 to 0.1%, preferably 0.0005 to 0.1%, more preferably 0.004 to 0.05%, still more preferably 0.007 to 0.01%, in terms of the total amount based on the weight of the crude glycerol. Manganese, copper, and zinc may be present in an amount of from 0.000005 to 0.01%, preferably 0.000007 to 0.005%, more preferably 0.00001 to 0.001%, in terms of the total amount.

The purity of the crude glycerol may be 10% or higher, preferably 50% or higher, more preferably 70% or higher, particularly preferably 80% or higher. So long as the amount of the impurities is kept within the aforementioned range, the purity of the glycerol may be 90% or higher.

Crude glycerol is produced in the production of biodiesel fuel, and when used as the carbon source in fermentation, will enable production of more L-amino acid as compared to when using an equal weight of reagent glycerol. To “produce more L-amino acid as compared to reagent glycerol” means to increase the amino acid production amount by 5% or more, preferably 10% or more, more preferably 20% or more, as compared to when reagent glycerol is used as the carbon source. The “reagent glycerol” means glycerol sold as regent grade, or glycerol with a purity which is equivalent to the purity of glycerol sold as regent grade. Reagent glycerol preferably is 99% pure by weight or higher, and pure glycerol is particularly preferred. The “reagent glycerol of the same amount as that of crude glycerol” means reagent glycerol of the same weight as the crude glycerol except for water, when the crude glycerol contains water.

The crude glycerol may be diluted with a solvent such as water, however, the above descriptions concerning the amounts of glycerol and impurities are applied to the crude glycerol before dilution. That is, when crude glycerol contains a solvent, and when the solvent is eliminated so that the solvent is 30% by weight or less, preferably 20% by weight or less, more preferably 10% by weight or less, if the amount of the impurities is within the aforementioned ranges, then the glycerol is considered crude glycerol.

### <2> Bacteria

Bacteria belonging to the family Enterobacteriaceae and which are able to produce an L-amino acid are used. The Enterobacteriaceae family encompasses bacteria belonging to the genera of Escherichia, Enterobacter, Erwinia, Klebsiella, Pantoea, Photorhabdus, Providencia, Salmonella, Serratia, Shigella, Morganella, Yersinia, and so forth. In particular, bacteria classified into the family Enterobacteriaceae defined by the taxonomy used by the NCBI (National Center for Biotechnology Information) database are preferred.

A “bacterium belonging to the genus Escherichia” or “Escherichia bacteria” means that the bacterium is classified into the genus Escherichia according to the classification known to a person skilled in the art of microbiology, although the bacterium is not particularly limited to these. An example of a bacterium belonging to the genus Escherichia is Escherichia coli (E. coli).

Further examples include the bacteria described in the work of Neidhardt et al. (Neidhardt F. C. Ed., 1996, Escherichia coli and Salmonella: Cellular and Molecular Biology/Second Edition, pp. 2477-2483, Table 1, American Society for Microbiology Press, Washington, D.C.). Specific examples include Escherichia coli W3110 (ATCC 27325), Escherichia coli MG1655 (ATCC 47076) derived from the prototype wild-type K12 strain, and so forth.

These strains are available from, for example, the American Type Culture Collection (Address: P.O. Box 1549, Manassas, Va. 20108, United States of America). That is, accession numbers are given to each of the strains, and the strains can be ordered using these numbers. The accession numbers of the strains are listed in the catalogue of the American Type Culture Collection.

A “bacterium belonging to the genus Pantoea” or “Pantoea bacterium” means that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii or the like, based on the nucleotide sequence analysis of 16S rRNA, etc. (Int. J. Syst. Bacteriol., 43, 162-173 (1993)). Bacteria belonging to the genus Pantoea encompass these bacteria re-classified into the genus Pantoea as described above.

In order to enhance glycerol assimilation in the bacteria, expression of the glpR gene (EP 1715056) may be attenuated, or expression of the glycerol metabolism genes (EP 1715055 A), such as glpA, glpB, glpC, glpD, glpE, glpF, glpG, glpK, glpQ, glpT, glpX, tpiA, gldA, dhaK, dhaL, dhaM, dhaR, fsa and talC, may be enhanced.

The “bacterium having an L-amino acid-producing ability” or the “bacterium which is able to produce an L-amino acid” means a bacterium which can produce and secrete an L-amino acid into a medium when it is cultured in the medium. It preferably means a bacterium which can cause accumulation of an desired L-amino acid in the medium in an amount not less than 0.5 g/L, more preferably not less than 1.0 g/L. The term “L-amino acid” encompasses L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. L-threonine, L-lysine, L-glutamic acid, and L-tryprophan are especially preferred.

Hereinafter, methods for imparting an L-amino acid-producing ability to such bacteria as described above, or methods for enhancing an L-amino acid-producing ability of such bacteria as described above are described.

Methods which have been conventionally employed in the breeding of coryneform bacteria or Escherichia bacteria (see “Amino Acid Fermentation”, Gakkai Shuppan Center (Ltd.), 1st Edition, published May 30, 1986, pp. 77-100) can be used to impart the ability to produce L-amino acids. These methods include imparting properties such as an auxotrophic mutant, resistance to an L-amino acid analogue, or a metabolic regulation mutant, or by constructing a recombinant strain with increased expression of an L-amino acid biosynthetic enzyme. In the breeding of an L-amino acid-producing bacteria, one or more of these properties may be imparted. The expression of L-amino acid biosynthetic enzyme(s) can be increased singly or in combinations of two or more. Furthermore, the methods of imparting properties such as an auxotrophic mutation, analogue resistance, or metabolic regulation mutation may be combined with the technique of enhancing the expression of the biosynthetic enzymes.

An auxotrophic mutant strain, L-amino acid analogue-resistant strain, or metabolic regulation mutant strain with the ability to produce an L-amino acid can be obtained by subjecting a parent or wild-type bacterial strain to conventional mutatagenesis, such as by exposing the bacteria to X-rays or UV irradiation, or by treating the bacteria with a mutagen such as N-methyl-N′-nitro-N-nitrosoguanidine, etc., and then selecting the bacteria which have the desired property, such as autotrophy, analogue resistance, or a metabolic regulation mutation, and which also are able to produce an L-amino acid.

Moreover, imparting or enhancing the ability to produce an L-amino acid can also be attained by increasing enzymatic activity by genetic recombination. Enzymatic activity can be increased, for example, by modifying the bacterium to increase the expression of a gene encoding an enzyme involved in the biosynthesis of the desired L-amino acid. To increase the expression of the desired gene, an amplification plasmid containing the gene can be introduced into an appropriate plasmid, for example, a plasmid vector containing at least a gene responsible for replication and proliferation of the plasmid in the microorganism. Other methods to increase the expression of the desired gene include by increasing the copy number of the gene on the chromosome by conjugation, transfer or the like, or by introducing a mutation into the promoter region of the gene (refer to International Patent Publication WO95/34672).

When the objective gene is introduced into an amplification plasmid or the chromosome, any promoter may be used to express the gene so long as the chosen promoter functions in bacteria of the Enterobacteriaceae family. The promoter may be the native promoter for the desired gene, or may be modified. The expression can also be controlled by choosing a promoter that is particularly potent in bacteria of the Enterobacteriaceae family, or by making the −35 and −10 regions of the promoter closer to the consensus sequence. These are described in International Patent Publication WO00/18935, European Patent Publication No. 1010755, and so forth.

Methods for imparting the ability to produce an L-amino acid to bacteria, and bacteria imparted with L-amino acid-producing ability, are exemplified below.

L-Threonine-Producing Bacteria

Preferred L-theonine producing microorganisms include bacteria that have increased activity/activities of one or more enzymes of the L-threonine biosynthesis system. Examples of the L-threonine biosynthetic enzymes include aspartokinase III gene (lysC), aspartate semialdehyde dehydrogenase (asd), aspartokinase I (thrA), homoserine kinase (thrB), and threonine synthase (thrC), which are all encoded by the threonine operon, and aspartate aminotransferase (aspartate transaminase) (aspC). The name of the gene encoding each enzyme is stated in parentheses after the enzyme's name, and this convention is seen throughout the specification. Aspartate semialdehyde dehydrogenase, aspartokinase I, homoserine kinase, aspartate aminotransferase, and threonine synthase are particularly preferred. The genes encoding the L-threonine biosynthetic enzymes may be introduced into an Escherichia bacterium which has been modified to decrease threonine decomposition, such as the TDH6 strain, which is deficient in threonine dehydrogenase activity (JP 2001-346578 A).

L-threonine biosynthetic enzyme activity is inhibited by the end-product, L-threonine. Therefore, these enzymes are preferably modified so that they are desensitized to feedback inhibition by L-threonine. The thrA, thrB and thrC genes constitute the threonine operon, and the threonine operon forms an attenuator structure. The expression of the threonine operon is inhibited by isoleucine and threonine which are present in the culture medium, and is also suppressed by attenuation. Therefore, the threonine operon is preferably modified by removing the leader sequence or attenuator in the attenuation region (refer to Lynn, S. P., Burton, W. S., Donohue, T. J., Gould, R. M., Gumport, R. L, and Gardner, J. F., J. Mol. Biol. 194:59-69 (1987); WO02/26993; WO2005/049808).

The native promoter of the threonine operon is located upstream of the threonine operon, and may be replaced with a non-native promoter (refer to WO98/04715). Alternatively, the threonine operon may be altered so that expression of the threonine biosynthesis gene(s) is controlled by the repressor and promoter of λ-phage (EP 0593792). Furthermore, to desensitize the bacterium to feedback inhibition by L-threonine, a strain resistant to α-amino-β-hydroxyisovaleric acid (AHV) may be selected.

It is preferable to increase the copy number of the above-described modified threonine operon in the host bacterium, or to increase expression of the modified operon by ligating it to a more potent promoter. The copy number can also be increased by, besides amplification using a plasmid, transferring the threonine operon to the genome using a transposon or Mu-phage.

Besides increasing expression of the L-threonine biosynthetic genes, expression of the genes involved in the glycolytic pathway, TCA cycle, or respiratory chain can be increased. Also, expression of the genes that regulate the expression of these genes, or the genes involved in sugar uptake can also be increased. Examples of genes that are effective for L-threonine production include the transhydrogenase gene (pntAB, EP 733712 B), phosphoenolpyruvate carboxylase gene (pepC, WO95/06114), phosphoenolpyruvate synthase gene (pps, EP 877090 B), and pyruvate carboxylase gene derived from coryneform bacterium or Bacillus bacterium (WO99/18228, EP 1092776 A).

It is also preferable to increase expression of a gene that imparts L-threonine or L-homoserine resistance, or both, to the host. Examples of the genes that impart resistance include rhtA (Res. Microbiol., 154:123-135 (2003)), rhtB (EP 0994190 A), rhtC (EP 1013765 A), yfiK, and yeaS (EP 1016710 A). To impart L-threonine resistance to the host, the methods described in EP 0994190 A and WO90/04636 can be used.

L-threonine-producing bacteria, and parent strains which can be used to derive such bacteria, include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and so forth.

The TDH-6 strain is deficient in the thrC gene, as well as being sucrose-assimilative, and the ilvA gene has a leaky mutation. This strain also has a mutation in the rhtA gene, which imparts resistance to high concentrations of threonine or homoserine. The B-3996 strain contains the plasmid pVIC40, which was obtained by inserting the thrA*BC operon (the thrA gene is mutated) into a RSF1010-derived vector. The mutant thrA gene encodes aspartokinase homoserine dehydrogenase I which is substantially desensitized to feedback inhibition by threonine. The B-3996 strain was deposited on Nov. 19, 1987 in the All-Union Scientific Center of Antibiotics (Nagatinskaya Street 3-A, 117105 Moscow, Russia) under the accession number RIA 1867. This strain was also deposited at the Russian National Collection of Industrial Microorganisms (VKPM) (1 Dorozhny proezd., 1 Moscow 117545, Russia) on Apr. 7, 1987 under the accession number VKPM B-3996.

E. coli VKPM B-5318 (EP 0593792 B) may also be used to derive an L-threonine-producing bacterium. The B-5318 strain is prototrophic with regard to isoleucine, and a temperature-sensitive lambda-phage Cl repressor and PR promoter replace the regulatory region of the threonine operon in pVIC40. The VKPM B-5318 strain was deposited at the Russian National Collection of Industrial Microorganisms (VKPM) (1 Dorozhny proezd., 1 Moscow 117545, Russia) on May 3, 1990 under an accession number of VKPM B-5318.

The Escherichia coli thrA gene which encodes aspartokinase homoserine dehydrogenase I has been elucidated (nucleotide positions 337 to 2799, GenBank accession NC_{—}000913.2, gi: 49175990). The thrA gene is located between the thrL and thrB genes on the chromosome of E. coli K-12. The Escherichia coli thrB gene which encodes homoserine kinase has been elucidated (nucleotide positions 2801 to 3733, GenBank accession NC 000913.2, gi: 49175990). The thrB gene is located between the thrA and thrC genes on the E. coli K-12 chromosome. The Escherichia coli thrC gene which encodes threonine synthase has been elucidated (nucleotide positions 3734 to 5020, GenBank accession NC 000913.2, gi: 49175990). The thrC gene is located between the thrB gene and the yaaX open reading frame on the E. coli K-12 chromosome. All three genes function as a single threonine operon. To increase expression of the threonine operon, the attenuator region which negatively affects transcription can be removed from the operon (WO2005/049808, WO2003/097839).

The mutant thrA gene as described above, as well as the thrB and thrC genes, can be obtained as one operon from the well-known plasmid pVIC40, which is present in the threonine-producing E. coli strain VKPM B-3996. pVIC40 is described in detail in U.S. Pat. No. 5,705,371.

The rhtA gene is located at 18 min on the E. coli chromosome, close to the glnHPQ operon. This operon encodes components of the glutamine transport system. The rhtA gene is identical to ORF1 (ybiF gene, nucleotide positions 764 to 1651, GenBank accession number AAA218541, gi:440181) and is located between the pexB and ompX genes. The unit expressing a protein encoded by ORF1 has been designated the rhtA gene (rht: resistance to homoserine and threonine). Also, the rhtA23 mutation is an A-for-G substitution at position −1 with respect to the ATG start codon (ABSTRACTS of the 17th International Congress of Biochemistry and Molecular Biology in conjugation with Annual Meeting of the American Society for Biochemistry and Molecular Biology, San Francisco, Calif. Aug. 24-29, 1997, abstract No. 457, EP 1013765 A).

The E. coli asd gene has already been elucidated (nucleotide positions 3572511 to 3571408, GenBank accession NC_{—}000913.1, gi: 16131307), and can be obtained by PCR (polymerase chain reaction; refer to White, T. J. et al., Trends Genet, 5, 185 (1989)) utilizing primers prepared based on the nucleotide sequence of the gene. The asd genes from other microorganisms can also be obtained in a similar manner.

Also, the E. coli aspC gene has already been elucidated (nucleotide positions 983742 to 984932, GenBank accession NC 000913.1, gi:16128895), and can be obtained by PCR. The aspC genes from other microorganisms can also be obtained in a similar manner.

L-Lysine-Producing Bacteria

Examples of L-lysine-producing Escherichia bacteria include mutants which are resistant to an L-lysine analogue. The L-lysine analogue inhibits growth of the Escherichia bacteria, but this inhibition is fully or partially desensitized when L-lysine is present in the medium. Examples of the L-lysine analogue include, but are not limited to, oxalysine, lysine hydroxamate, S-(2-aminoethyl)-L-cysteine (AEC), γ-methyllysine, α-chlorocaprolactam, and so forth. Mutants having resistance to these lysine analogues can be obtained by subjecting the Escherichia bacteria to conventional artificial mutagenesis. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The WC196 strain is an L-lysine-producing Escherichia coli bacterium. This bacterial strain was bred by conferring AEC resistance to the W3110 strain, which was derived from Escherichia coli K-12. The resulting strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of L-lysine-producing bacteria, and parent strains which can be used to derive L-lysine-producing bacteria, also include strains in which expression is increased of one or more genes encoding an L-lysine biosynthetic enzyme. Examples of such enzymes include, but are not limited to, dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), diaminopimelate epimerase (dapF), tetrahydrodipicolinate succinylase (dapD), succinyl diaminopimelate deacylase (dapE), and aspartase (aspA) (EP 1253195 A). Dihydrodipicolinate reductase, diaminopimelate decarboxylase, diaminopimelate dehydrogenase, phosphoenolpyrvate carboxylase, aspartate aminotransferase, diaminopimelate epimerase, aspartate semialdehyde dehydrogenease, tetrahydrodipicolinate succinylase, and succinyl diaminopimelate deacylase are especially preferred. In addition, the parent strains may have increased expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

L-lysine-producing bacteria, and parent strains which can be used to derive L-lysine-producing bacteria, also include strains which have been modified to decrease or eliminate the activity of an enzyme that catalyzes a reaction which results in a compound other than L-lysine via a biosynthetic pathway which branches off from the pathway of L-lysine. Examples of these enzymes include homoserine dehydrogenase, lysine decarboxylase (U.S. Pat. No. 5,827,698), and the malic enzyme (WO2005/010175).

Preferred examples of L-lysine producing strains include E. coli WC196ΔcadAΔldc/pCABD2 (WO2006/078039). This strain was obtained by introducing the plasmid pCABD2, which is disclosed in U.S. Pat. No. 6,040,160, into the WC1-96 strain, in which the cadA and ldcC genes encoding lysine decarboxylase are disrupted. pCABD2 contains a mutant Escherichia coli dapA gene encoding dihydrodipicolinate synthase (DDPS) desensitized to feedback inhibition by L-lysine, a mutant Escherichia coli lysC gene encoding aspartokinase III desensitized to feedback inhibition by L-lysine, the Escherichia coli dapB gene encoding dihydrodipicolinate reductase, and the ddh gene derived from Brevibacterium lactofermentum encoding diaminopimelate dehydrogenase.

L-Cysteine-Producing Bacteria

L-cysteine-producing bacteria, and parent strains which can be used to derive L-cysteine-producing bacteria, include, but are not limited to, Escherichia bacteria, such as E. coli JM15, which is transformed with different cysE alleles encoding feedback-resistant serine acetyltransferases (U.S. Pat. No. 6,218,168, Russian patent application 2003121601); E. coli W3110 which over-expresses genes which encode proteins which promote secretion of substances which are toxic for cells (U.S. Pat. No. 5,972,663); E. coli strains with decreased cysteine desulfohydrase activity (JP 11155571 A2); E. coli W3110 with increased activity of a positive transcriptional regulator for the cysteine regulon encoded by the cysB gene (WO0/27307A1), and so forth.

L-Leucine-Producing Bacteria

L-leucine-producing bacteria, and parent strains which can be used to derive L-leucine-producing bacteria, include, but are not limited to, Escherichia strains, such as E. coli strains resistant to leucine (for example, the strain 57 (VKPM B-7386, U.S. Pat. No. 6,124,121)) or leucine analogues including β-2-thienylalanine, 3-hydroxyleucine, 4-azaleucine, 5,5,5-trifluoroleucine, and so forth (JP 62-34397 B and JP 8-70879 A); E. coli strains obtained by the genetic engineering method described in WO96/06926; E. coli H-9068 (JP 8-70879 A), and so forth.

The bacteria may be improved by increasing expression of one or more genes involved in L-leucine biosynthesis. Examples include the genes of the leuABCD operon, which may include a mutant leuA gene encoding isopropyl malate synthase desensitized to feedback inhibition by L-leucine (U.S. Pat. No. 6,403,342). In addition, the bacteria may be improved by increasing expression of one or more genes encoding proteins which promote secretion of the L-amino acid from the bacterial cell. Examples of such genes include b2682 and b2683 (ygaZH genes) (EP 1239041 A2).

L-Histidine-Producing Bacteria

L-histidine-producing bacteria, and parent strains which can be used to derive L-histidine-producing bacteria include, but are not limited to, Escherichia strains, such as E. coli strain 24 (VKPM B-5945, RU2003677), E. coli strain 80 (VKPM B-7270, RU2119536), E. coli NRRL B-12116-B 12121 (U.S. Pat. No. 4,388,405), E. coli H-9342 (FERM BP-6675) and H-9343 (FERM BP-6676) (U.S. Pat. No. 6,344,347), E. coli H-9341 (FERM BP-6674) (EP 1085087), E. coli AI80/pFM201 (U.S. Pat. No. 6,258,554), and so forth.

L-histidine-producing bacteria, and parent strains which can be used to derive L-histidine-producing bacteria, also include strains in which expression is increased of one or more genes encoding an L-histidine biosynthetic enzyme. Examples of such genes include the genes encoding ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the L-histidine biosynthetic enzymes encoded by the hisG and hisBHAFI genes are inhibited by L-histidine, and therefore the ability to produce L-histidine can also be efficiently enhanced by introducing a mutation which confers resistance to feedback inhibition into the gene encoding ATP phosphoribosyltransferase (hisG) (Russian Patent Nos. 2003677 and 2119536).

Specific examples of strains that are able to produce L-histidine include E. coli FERM-P 5038 and 5048 which have been transformed with a vector carrying a DNA encoding an L-histidine-biosynthetic enzyme (JP 56-005099 A), E. coli strains transformed with a gene which promotes amino acid export (EP 1016710 A), E. coli 80 strain which is resistant to sulfaguanidine, DL-1,2,4-triazole-3-alanine, and streptomycin (VKPM B-7270, Russian Patent No. 2119536), and so forth.

L-Glutamic Acid-Producing Bacteria

L-glutamic acid-producing bacteria, and parent strains which can be used to derive L-glutamic acid-producing bacteria, include, but are not limited to, Escherichia strains, such as E. coli VL334thrC⁺ (EP 1172433). E. coli VL334 (VKPM B-1641) is an L-isoleucine and L-threonine auxotrophic strain having mutations in the thrC and ilvA genes (U.S. Pat. No. 4,278,765). A wild-type allele of the thrC gene was transferred by transduction using a bacteriophage PI grown on the wild-type E. coli strain K12 (VKPM B-7) cells. As a result, the L-isoleucine auxotrophic strain VL334thrC⁺ (VKPM B-8961), which is able to produce L-glutamic acid, was obtained.

L-glutamic acid-producing bacteria, and parent strains which can be used to derive L-glutamic acid-producing bacteria, include, but are not limited to, strains in which expression is increased of one or more genes encoding an L-glutamic acid biosynthetic enzyme. Examples of such genes include the genes encoding glutamate dehydrogenase (gdhA), glutamine synthetase (glnA), glutamate synthetase (gltAB), isocitrate dehydrogenase (icdA), aconitate hydratase (acnA, acnB), citrate synthase (gltA), methyl citrate synthase gene (prpC), phosphoenolpyruvate carboxylase (ppc), pyruvate dehydrogenase (aceEF, lpdA), pyruvate kinase (pykA, pykF), phosphoenolpyruvate synthase (ppsA), enolase (eno), phosphoglyceromutase (pgmA, pgmI), phosphoglycerate kinase (pgk), glyceraldehyde-3-phosphate dehydrogenase (gapA), triose phosphate isomerase (tpiA), fructose bisphosphate aldolase (fbp), phosphofructokinase (pfkA, pfkB), glucose phosphate isomerase (pgi), and so forth. Glutamate dehydrogenase, citrate synthase, phosphoenolpyruvate carboxylase, and methyl citrate synthase are preferred.

Examples of strains modified so that expression is increased of the citrate synthetase gene, the phosphoenolpyruvate carboxylase gene, and/or the glutamate dehydrogenase gene include those disclosed in EP 1078989 A, EP 955368 A, and EP 952221 A.

L-glutamic acid-producing bacteria, and parent strains which can be used to derive L-glutamic acid-producing bacteria, also include strains which have been modified to decrease or eliminate activity of an enzyme that catalyzes synthesis of a compound other than L-glutamic acid via a pathway which branches off from the L-glutamic acid biosynthesis pathway. Examples of such enzymes include isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), glutamate decarboxylase (gadAB), and so forth. Escherichia bacteria with no α-ketoglutarate dehydrogenase activity, or a decreased amount, and methods for obtaining them are described in U.S. Pat. Nos. 5,378,616 and 5,573,945.

Specifically, these strains include the following:

E. coli W3110sucA::Km^{r}

E. coli AJ12624 (FERM BP-3853)

E. coli AJ12628 (FERM BP-3854)

E. coli AJ12949 (FERM BP-4881)

E. coli W3110sucA::Km^{r} is obtained by disrupting the α-ketoglutarate dehydrogenase gene (hereinafter also referred to as the “sucA gene”) of E. coli W3110. This strain is completely deficient in α-ketoglutarate dehydrogenase.

Other examples of L-glutamic acid-producing bacteria include Escherichia bacteria which are resistant to an aspartic acid antimetabolite. These strains can also be deficient in α-ketoglutarate dehydrogenase activity and include, for example, E. coli AJ13199 (FERM BP-5807) (U.S. Pat. No. 5,908,768), FFRM P-12379, which additionally has a decreased ability to decompose L-glutamic acid (U.S. Pat. No. 5,393,671); AJ13138 (FERM BP-5565) (U.S. Pat. No. 6,110,714), and so forth.

An example of an L-glutamic acid producing strain of Pantoea ananatis is Pantoea ananatis AJ13355. This strain was isolated from soil in Iwata-shi, Shizuoka-ken, Japan, and can proliferate in a medium containing L-glutamic acid and a carbon source at a low pH. The Pantoea ananatis AJ13355 strain was deposited at the National Institute of Advanced Industrial Science and Technology, International Patent Organism Depositary (Tsukuba Central 6, 1-1, Higashi 1-Chome, Tsukuba-shi, Ibaraki-ken, 305-8566, Japan) on Feb. 19, 1998 and received an accession number of FERM P-16644. It was then converted to an international deposit under the provisions of Budapest Treaty on Jan. 11, 1999 and received an accession number of FERM BP-6614. This strain was identified as Enterobacter agglomerans when it was isolated, and was deposited as the Enterobacter agglomerans AJ13355 strain. However, it was recently re-classified as Pantoea ananatis on the basis of nucleotide sequencing of 16S rRNA and so forth.

Furthermore, another L-glutamic acid producing Pantoea ananatis strain is Pantoea bacteria with no α-ketoglutarate dehydrogenase (αKGDH) activity, or a reduced amount. Examples include the AJ13356 strain (U.S. Pat. No. 6,331,419) which is the AJ13355 strain with no αKGDH-E1 subunit gene (sucA), and the SC17sucA strain (U.S. Pat. No. 6,596,517) which is deficient in the sucA gene, and is derived from the SC17 strain, which was selected as a low phlegm production mutant strain. The AJ13356 strain was deposited at the National Institute of Bioscience and Human-Technology, Agency of Industrial Science and Technology, Ministry of International Trade and Industry (currently, the independent administrative agency, National Institute of Advanced Industrial Science and Technology, International Patent Organism Depositary (Tsukuba Central 6, 1-1, Higashi 1-Chome, Tsukuba-shi, Ibaraki-ken, Japan, postal code: 305-8566)) on Feb. 19, 1998, and assigned an accession number of FERM P-16645. Then, the deposit was converted to an international deposit under the provisions of the Budapest Treaty on Jan. 11, 1999, and assigned an accession number of FERM BP-6616. Although the AJ13355 and AJ13356 strains were deposited at the aforementioned depository as Enterobacter agglomerans, they are referred to as Pantoea ananatis in this specification. The SC17sucA strain was assigned a private number of AJ417, and deposited at the National Institute of Advanced Industrial Science and Technology, International Patent Organism Depositary on Feb. 26, 2004, under an accession number of FERM BP-08646.

Examples of L-glutamic acid-producing Pantoea ananatis strains further include SC17sucA/RSFCPG+pSTVCB, AJ13601, NP106, and NA1. The SC17sucA/RSFCPG+pSTVCB strain is obtained by transformation of SC17sucA with the plasmid RSFCPG containing the Escherichia coli genes encoding citrate synthase (gltA), phosphoenolpyruvate carboxylase (ppsA), and glutamate dehydrogenase (gdhA), and the plasmid pSTVCB containing the gene encoding citrate synthase (gltA) derived from Brevibacterium lactofermentum. The AJ13601 strain was selected from the SC17sucA/RSFCPG+pSTVCB strain for its resistance to high concentrations of L-glutamic acid at a low pH. Furthermore, the NP106 strain corresponds to the AJ13601 strain with no plasmid RSFCPG+pSTVCB, as described in the examples section. The AJ13601 strain was deposited at the National Institute of Advanced Industrial Science and Technology, International Patent Organism Depositary on Aug. 18, 1999, and assigned an accession number FERM P-17516. Then, the deposit was converted into an international deposit under the provisions of the Budapest Treaty on Jul. 6, 2000, and assigned an accession number FERM BP-7207.

L-Phenylalanine-Producing Bacteria

L-phenylalanine-producing bacteria, and parent strains which can be used to derive L-phenylalanine-producing bacteria, include, but are not limited to, Escherichia strains, such as E. coli AJ12739 (tyrA::Tn10, tyrR) (VKPM B-8197) deficient in chorismate mutase, prephenate dehydrogenase, and the tyrosine repressor (WO03/044191); E. coli HW1089 (ATCC 55371) which contains a mutant pheA34 gene encoding chorismate mutase and prephenate dehydratase desensitized to feedback inhibition (U.S. Pat. No. 5,354,672); E. coli MWEC10′-b (KR8903681); and E. coli NRRL B-12141, NRRL B-12145, NRRL B-12146 and NRRL B-12147 (U.S. Pat. No. 4,407,952). Also, E. coli K-12 [W3110(tyrA)/pPHAB (FERM BP-3566) with genes encoding chorismate mutase and prephenate dehydratase desensitized to feedback inhibition, E. coli K-12 [W3110(tyrA)/pPHAD] (FERM BP-12659), E. coli K-12 [W3110(tyrA)/pPHATerm] (FERM BP-12662) and E. coli K-12 [W3110(tyrA)/pBR-aroG4, pACMAB] also known as AJ12604 (FERM BP-3579) may be used to derive L-phenylalanine producing bacteria (EP 488-424 B1). Furthermore, L-phenylalanine-producing Escherichia bacteria with increased activity of the protein encoded by the yedA gene or the yddG gene may also be used (U.S. Patent Published Applications Nos. 2003/0148473 A1 and 2003/0157667 A1, WO03/044192).

L-Tryptophan-Producing Bacteria

L-tryptophan-producing bacteria, and parent strains which can be used to derive L-tryptophan-producing bacteria, include, but are not limited to, Escherichia strains, such as E. coli JP4735/pMU3028 (DSM10122) and JP6015/pMU91 (DSM10123) deficient in the tryptophanyl-tRNA synthetase encoded by a mutant trpS gene (U.S. Pat. No. 5,756,345); E. coli SV164 (pGH5) with a serA allele encoding phosphoglycerate dehydrogenase not subject to feedback inhibition by serine and a trpE allele encoding anthranilate synthase not subject to feedback inhibition by tryptophan (U.S. Pat. No. 6,180,373); E. coli AGX17 (pGX44) (NRRL B-12263) and AGX6(pGX50)aroP (NRRL B-12264) deficient in tryptophanase (U.S. Pat. No. 4,371,614); E. coli AGX17/pGX50, pACKG4-pps in which the ability to produce phosphoenolpyruvate is increased (WO9708333, U.S. Pat. No. 6,319,696), and so forth. L-Tryptophan-producing bacteria belonging to the genus Escherichia with an enhanced activity of the protein encoded by the yedA gene or the yddG gene may also be used (U.S. Patent Published Application Nos. 2003/0148473 A1 and 2003/0157667 A1).

L-tryptophan-producing bacteria, and parent strains which can be used to derive L-tryptophan-producing bacteria, also include strains in which one or more activities are enhanced of the following enzymes: anthranilate synthase (trpE), phosphoglycerate dehydrogenase (serA), 3-deoxy-D-arabinoheptulosonate-7-phosphate synthase (aroG), 3-dehydroquinate synthase (aroB), shikimate dehydrogenase (aroE), shikimate kinase (aroL), 5-enolpyruvylshikimate-3-phosphate synthase (aroA), chorismate synthase (aroC), prephenate dehydratase, chorismate mutase, and tryptophan synthase (trpAB). Prephenate dehydratase and chorismate mutase are encoded by the pheA gene as a bifunctional enzyme (CM-PD). Phosphoglycerate dehydrogenase, 3-deoxy-D-arabinoheptulosonate-7-phosphate synthase, 3-dehydroquinate synthase, shikimate dehydratase, shikimate kinase, 5-enolpyruvylshikimate-3-phosphate synthase, chorismate synthase, prephenate dehydratase, and chorismate mutase-prephenate dehydratase are especially preferred. The anthranilate synthase and phosphoglycerate dehydrogenase both are subject to feedback inhibition by L-tryptophan and L-serine, and therefore a mutation desensitizing this inhibition may be introduced into these enzymes. Specific examples of strains having such a mutation include E. coli SV164 which harbors desensitized anthranilate synthase, and a transformant strain obtained by introducing into E. coli SV164 the plasmid pGH5 (WO94/08031), which contains a mutant serA gene encoding feedback-desensitized phosphoglycerate dehydrogenase.

L-tryptophan-producing bacteria, and parent strains which can be used to derive L-tryptophan-producing bacteria, also include strains transformed with the tryptophan operon which contains a gene encoding inhibition-desensitized anthranilate synthase (JP 57-71397 A, JP 62-244382 A, U.S. Pat. No. 4,371,614). Moreover, L-tryptophan-producing ability may be imparted by increasing expression of the gene which encodes tryptophan synthase in the tryptophan operon (trpBA). Tryptophan synthase consists of α and β subunits, which are encoded by trpA and trpB, respectively. In addition, L-tryptophan-producing ability may be improved by increasing expression of the isocitrate lyase-malate synthase operon (WO2005/103275).

L-Proline-Producing Bacteria

L-proline-producing bacteria, and parent strains which can be used to derive L-proline-producing bacteria, include, but are not limited to, Escherichia strains, such as E. coli 702ilvA (VKPM B-8012) which is deficient in the ilvA gene and is able to produce L-proline (EP 1172433).

The bacteria may be improved by increasing the expression of one or more genes involved in L-proline biosynthesis. Examples of such genes include the proB gene encoding glutamate kinase which is desensitized to feedback inhibition by L-proline (DE Patent 3127361). In addition, the bacteria may be improved by increasing the expression of one or more genes encoding proteins which promote secretion of an L-amino acid from the bacterial cell. Such genes are exemplified by b2682 and b2683 (ygaZH genes) (EP 1239041 A2).

Examples of Escherichia bacteria which are able to produce L-proline include the following E. coli strains: NRRL B-12403 and NRRL B-12404 (GB Patent 2075056), VKPM B-8012 (Russian patent application 2000124295), plasmid mutants described in DE Patent 3127361, plasmid mutants described by Bloom F. R. et al (The 15th Miami winter symposium, 1983, p. 34), and so forth.

L-Arginine-Producing Bacteria

L-arginine-producing bacteria, and parent strains which can be used to derive L-arginine-producing bacteria, include, but are not limited to, Escherichia strains, such as E. coli strain 237 (VKPM B-7925) (U.S. Patent Published Application No. 2002/058315 A1) and its derivative strains with mutant N-acetylglutamate synthase (Russian Patent Application No. 2001112869), E. coli strain 382 (VKPM B-7926) (EP 1170358A1), and an arginine-producing strain with the argA gene encoding N-acetylglutamate synthetase (EP 1170361 A1).

L-arginine-producing bacteria, and parent strains which can be used to derive L-arginine-producing bacteria, also include strains in which expression is increased of one or more genes encoding an L-arginine biosynthetic enzyme. Examples of such genes include the genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase (carAB).

L-Valine-Producing Bacteria

L-valine-producing bacteria, and parent strains which can be used to derive L-valine-producing bacteria, include, but are not limited to, strains which have been modified to overexpress the ilvGMEDA operon (U.S. Pat. No. 5,998,178). The region of the ilvGMEDA operon which is required for attenuation can be removed so that expression of the operon is not attenuated by the L-valine. Furthermore, the ilvA gene in the operon can be disrupted to decrease threonine deaminase activity.

L-valine-producing bacteria, and parent strains which can be used to derive L-valine-producing bacteria, also include mutants of amino-acyl t-RNA synthetase (U.S. Pat. No. 5,658,766). For example, E. coli VL1970, which has a mutation in the ileS gene encoding isoleucine tRNA synthetase, can be used. E. coli VL1970 has been deposited at the Russian National Collection of Industrial Microorganisms (VKPM) (1 Dorozhny proezd., 1 Moscow 117545, Russia) on Jun. 24, 1988 under an accession number of VKPM B-4411.

Furthermore, mutants requiring lipoic acid for growth and/or lacking H⁺-ATPase can also be used as parent strains (WO96/06926).

L-Isoleucine-Producing Bacteria

L-isoleucine producing bacteria, and parent strains which can be used to derive L-isoleucine producing bacteria, include, but are not limited to, mutants which are resistant to 6-dimethylaminopurine (JP 5-304969 A), mutants which are resistant to an isoleucine analogue such as thiaisoleucine and isoleucine hydroxamate, and mutants which are additionally resistant to DL-ethionine and/or arginine hydroxamate (JP 5-130882 A). In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

When the L-amino acid-producing bacteria are bred using genetic recombination, the chosen genes are not limited to genes having the genetic information mentioned above, or to genes having known sequences, but genes with conservative mutations such as homologues or artificially modified genes can also be used so long as the functions of the encoded proteins are not degraded. That is, genes may be used which encode a known amino acid sequence but which contain one or more substitutions, deletions, insertions, additions, or the like of one or several amino acid residues at one or several positions.

Although the number of the “several” amino acid residues referred to herein may differ depending on the positions in the three-dimensional structure or types of amino acid residues in the protein, specifically, it may be preferably 1 to 20, more preferably 1 to 10, still more preferably 1 to 5. The conservative substitution is a mutation wherein substitution takes place mutually among Phe, Trp and Tyr, if the substitution site is an aromatic amino acid; among Leu, Ile and Val, if it is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid with a hydroxyl group. Typical examples of the conservative mutations are conservative substitutions, which include, specifically, substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Gly, Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val. The amino acid substitutions, deletions, insertions, additions, inversions or the like may result from a naturally-occurring mutation or variation due to individual differences or may be due to the difference of the microorganism species from which the genes are derived (mutant or variant). Such genes can be obtained by, for example, modifying the known nucleotide sequence of the gene by site-specific mutagenesis so that the amino acid residues at specific sites of the encoded protein include substitutions, deletions, insertions or additions of the amino acid residues.

Furthermore, such genes with a conservative mutation as mentioned above may encode a protein having a homology of 80% or more, preferably 90% or more, more preferably 95% or more, particularly preferably 97% or more, to the entire encoded amino acid sequence, and having a function equivalent to that of the wild-type protein.

Moreover, codons in the gene sequences may be replaced with other codons which are more easily used by the host into which the genes are introduced.

The genes with one or more conservative mutations may be obtained by methods typically used for mutagenesis, such as by treatment with mutagenesis agents.

Furthermore, the genes may contain DNA which can hybridize with a complementary sequence of the known gene sequence, or a probe which can be prepared from the complementary sequence under stringent conditions, and encodes a protein having a function equivalent to that of the known gene product. The “stringent conditions” are conditions under which a so-called specific hybrid is formed, and a non-specific hybrid is not formed. Examples of the stringent conditions include those under which highly homologous DNAs hybridize to each other, for example, DNAs not less than 80% homologous, preferably not less than 90% homologous, more preferably not less than 95% homologous, particularly preferably not less than 97% homologous, hybridize to each other, and DNAs less homologous than the above do not hybridize to each other, or conditions of washing once, preferably 2 or 3 times, at a salt concentration and temperature corresponding to washing which is typical of Southern hybridization, i.e., 1×SSC, 0.1% SDS at 60° C., preferably 0.1×SSC, 0.1% SDS at 60° C., more preferably 0.1×SSC, 0.1% SDS at 68° C.

As the probe, a part of the sequence which is complementary to the gene can also be used. The probe can be prepared by PCR using oligonucleotide primers prepared on the basis of the known gene sequence and a DNA fragment containing the nucleotide sequences as the template. For example, when a DNA fragment having a length of about 300 bp is used as the probe, washing conditions for hybridization may be 50° C., 2×SSC and 0.1% SDS.

### <3> Method for Producing L-Amino Acid

In the method for producing an L-amino acid of the present invention, an Enterobacteriaceae which is able to produce an L-amino acid is cultured in a medium containing glycerol as the carbon source to produce and cause accumulation of the L-amino acid in the culture, and the L-amino acid is collected from the culture medium.

The glycerol may be at any concentration so long as the chosen concentration is suitable for production of the L-amino acid. When glycerol is used as the sole carbon source in the medium, it should be present in the medium in an amount of preferably about 0.1 to 50% w/v, more preferably about 0.5 to 40% w/v, particularly preferably about 1 to 30% w/v %. Glycerol can also be used in combination with other carbon sources such as glucose, fructose, sucrose, blackstrap molasses, and starch hydrolysate. Although glycerol and other carbon sources may be mixed at an arbitrary ratio, the amount of glycerol in the carbon source should be 10% by weight or more, more preferably 50% by weight or more, still more preferably 70% by weight or more. Preferable other carbon sources are saccharides such as glucose, fructose, sucrose, lactose, galactose, blackstrap molasses, starch hydrolysate, a sugar solution obtained by hydrolysis of biomass, alcohols such as ethanol, and organic acids such as fumaric acid, citric acid and succinic acid. Glucose is preferred. Particularly preferred is a mixture of crude glycerol and glucose at a weight ratio of between 50:50 and 90:10, respectively.

Although the initial concentration of glycerol at the start of the culture is as described above, glycerol may be supplemented as it is consumed during the culture.

Crude glycerol can be added to the medium so that it is at a concentration which is within the ranges described above regarding the amount of glycerol, depending on purity of the glycerol. Furthermore, both glycerol and crude glycerol may be added to the medium.

Media which is conventionally used in the production of L-amino acids by fermentation using microorganisms can be used. That is, conventional media containing, besides a carbon source, a nitrogen source, inorganic ions, and optionally other organic components as required may be used. As the nitrogen source, inorganic ammonium salts such as ammonium sulfate, ammonium chloride, and ammonium phosphate, organic nitrogen such as soybean hydrolysate, ammonia gas, aqueous ammonia, and so forth may be used. As for organic trace nutrient sources, the medium should contain the required substances such as vitamin B₁, L-homoserine, and/or yeast extract or the like in appropriate amounts. Other than the above, potassium phosphate, magnesium sulfate, iron ions, manganese ions, and so forth are added in small amounts, as required. In addition, the medium may be either a natural or synthetic medium, so long as it contains a carbon source, a nitrogen source, inorganic ions, and other organic trace components as required.

The culture is preferably performed for 1 to 7 days under aerobic conditions. The culture temperature is preferably 24 to 45° C., and the pH during the culture is preferably between 5 and 9. To adjust the pH, inorganic, organic, acidic, or alkaline substances, ammonia gas, and so forth can be used. To collect the L-amino acid from the culture medium, a combination of known methods can be used, such as by using an ion exchange resin and precipitation. When the L-amino acid accumulates in the cells, supersonic waves, for example, or the like can be used to disrupt the cells, and the L-amino acid can be collected by using an ion exchange resin or the like, from the supernatant obtained by removing the cells from the cell-disrupted suspension by centrifugation.

### EXAMPLES

Hereinafter, the present invention will be more specifically explained with reference to the following non-limiting examples. In the examples, glycerol of reagent special grade (Nakalai Tesque) was used as reagent glycerol, and crude glycerols produced in biodiesel fuel production process (GLYREX, Nowit DCA-F and R Glycerin) were used as crude glycerol. As for the purity of these crude glycerols, the crude glycerol GLYREX had a purity of 86% by weight, the crude glycerol Nowit DCA-F had a purity of 79% by weight, and the crude glycerol R Glycerin had a purity of 78% by weight.

GLYREX was produced by FOX PETROLI (S.P.A. Sede legale e uffici, via Senigallia 29, 61100 Pesaro, Italy), and marketed by SVG (SVG ITALIA, SrL Via A. Majani, 2, 40122 Bologna (BO), Italy) as an animal feed additive. Nowit DCA-F is marketed by Nordische Oelwerke Walther Carrouxy GmbH & Co KG, Postfach 930247 Industriestrasse 61-65, 21107 Hamburg, Germany. Glycerin R is marketed by Inter-Harz GmbH, Postfach 1411 Rostock-Koppel 17, 25314 Elmshom, 25365 K1. Offenseth-Sparrieshoop, Germany.

### Example 1

### Growth of a Wild-Type Strain in Minimal Medium

The Escherichia coli MG1655 strain was cultured at 37° C. for 16 hours on LB agar medium (10 g/L of tryptone, 5 g/L of yeast extract, 10 g/L of NaCl, 15 g/L of agar), and the cells were scraped with a loop and suspended in a 0.9% NaCl solution. This suspension was inoculated into 5 ml of M9 medium (12.8 g/L of Na₂HPO₄.7H₂O, 0.6 g/L of K₂HPO₄, 0.5 g/L of NaCl, 1 g/L of NH₄Cl, 2 mM MgSO₄, 0.1 mM CaCl₂) containing 0.4% (w/v) either glucose, reagent glycerol, or crude glycerol as the carbon source, and the cells were cultured at 37° C. for 24 hours in a test tube.

This culture medium was diluted, and inoculated onto the LB agar medium, and the cells were cultured at 37° C. for 16 hours. In order to accurately measure the degree of growth, only viable cells were counted by colony formation. Averages of the results of the culture performed in test tubes in duplicate are shown in Table 1.

TABLE 1

| Carbon source | Viable cell count (per ml) |
|---|---|
| Glucose | 1.0 × 10⁵ |
| Reagent glycerol | 1.2 × 10⁵ |
| Crude glycerol GLYREX | 6.6 × 10⁶ |

The growth using the reagent glycerol was equivalent to or more than that observed with glucose. The growth with the crude glycerol was unexpectedly good, that is, 50 times or more as compared to that observed with glucose.

### Example 2

### L-Threonine Production

The Escherichia coli VKPM B-5318 strain, which is an L-threonine-producing bacterium, was cultured at 37° C. for 24 hours on LB agar medium (10 g/L of tryptone, 5 g/L of yeast extract, 10 g/L of NaCl, 15 g/L of agar) containing 20 mg/L of streptomycin sulfate. The cells on the agar medium were scraped, inoculated into 20 mL of L-threonine production medium containing 20 mg/L of streptomycin sulfate in a 500 ml-volume Sakaguchi flask, and cultured at 40° C. for 24 hours. The carbon source in the main culture was either glucose, reagent glycerol, crude glycerol, glucose and reagent glycerol at a ratio of 1:1, or glucose and crude glycerol at a ratio of 1:1. The total amount of the carbon source was 40 g/L for each of the sources.

| | |
|---|---|
| Group A: | |
| Carbon source | 40 g/L |
| MgSO₄•7H₂O | 1 g/L |
| Group B: | |
| Yeast extract | 2 g/L |
| FeSO₄•7H₂O | 10 mg/L |
| MnSO₄•4H₂O | 10 mg/L |
| KH₂PO₄ | 1 g/L |
| (NH₄)₂SO₄ | 16 g/L |
| Group C: | |
| Calcium carbonate | 30 g/L |

The components of Groups A and B were subjected to autoclave sterilization at 115° C. for 10 minutes, and the component of Group C was subjected to hot air sterilization at 180° C. for 3 hours. After the components of the three groups were cooled to room temperature, they were mixed into the media.

After completion of the culture, consumption of the added saccharide and glycerol was confirmed with BF-5 (Oji Scientific Instruments), and the degree of growth was measured by determining the turbidity (OD) at 600 nm. The amount of L-threonine was measured by liquid chromatography. Averages of the results of the culture performed in flasks in duplicate are shown in Table 2.

Under the main culture conditions, the amount of L-threonine was low when glucose was used as the carbon source. However, a marked improvement in the amount of L-threonine was observed when reagent glycerol was added alone or as a mixture. Furthermore, when crude glycerol was used alone, the increase in the L-threonine amount was higher than that observed when the reagent glycerol was used alone.

TABLE 2

| Carbon source | OD | Thr (g/l) |
|---|---|---|
| Glucose 40 g/l | 9.6 | 3.8 |
| Glucose 20 g/l + reagent | 11.6 | 12.3 |
| glycerol 20 g/l | | |
| Reagent glycerol 40 g/l | 10.1 | 9.9 |
| Glucose 20 g/l + crude | 11.2 | 12.5 |
| glycerol GLYREX 20 g/l | | |
| Crude glycerol GLYREX 40 g/l | 10.4 | 12.0 |

### Example 3

### L-Lysine Production Culture

The Escherichia coli WC196ΔcadAΔldc/pCABD2 strain, described in International Patent Publication WO2006/078039 (this strain is also called “WC196LC/pCABD2”), was used as an L-lysine-producing bacterium. The Escherichia coli WC196LC/pCABD2 was cultured at 37° C. for 24 hours on LB agar medium (10 g/L of tryptone, 5 g/L of yeast extract, 10 g/L of NaCl, 15 g/L of agar) containing 20 mg/L of streptomycin sulfate. The cells on the agar medium were scraped, inoculated into 20 mL of an L-lysine production medium containing 20 mg/L of streptomycin sulfate in a 500 ml-volume Sakaguchi flask, and cultured at 37° C. for 48 hours. The carbon source in the main culture was either glucose, reagent glycerol, crude glycerol, glucose and reagent glycerol at a ratio of 1:1, or glucose and the crude glycerol at a ratio of 1:1. The total amount of the carbon source was 40 g/L for all of the sources.

| | |
|---|---|
| Group A: | |
| Carbon source | 40 g/L |
| Group B: | |
| Yeast extract | 2 g/L |
| FeSO₄•7H₂O | 10 mg/L |
| MnSO₄•4H₂O | 10 mg/L |
| KH₂PO₄ | 1 g/L |
| (NH₄)₂SO₄ | 24 g/L |
| Group C: | |
| Calcium carbonate | 30 g/L |

The components of Groups A and B were subjected to autoclave sterilization at 115° C. for 10 minutes, and the component of Group C was subjected to hot air sterilization at 180° C. for 3 hours. After the components of the three groups were cooled to room temperature, they were mixed into the media.

After completion of the culture, consumption of the added saccharide and glycerol was confirmed with BF-5 (Oji Scientific Instruments), and the degree of growth was measured by determining the turbidity (OD) at 600 nm. The amount of L-lysine was measured with a Biotech Analyzer AS210 (Sakura Seiki). Averages of the results of the culture performed in flasks in duplicate are shown in Table 3.

Compared to the amount of L-lysine produced when glucose was used as the carbon source, the amount of L-lysine markedly decreased when reagent glycerol was used as a mixture or alone. However, when crude glycerol was used as a mixture or alone, the amount of L-lysine increased as compared to when reagent glycerol was used, and the amount of L-lysine is equivalent to the L-lysine amount obtained when using glucose as the carbon source.

TABLE 3

| Carbon source | OD | Lys (g/l) |
|---|---|---|
| Glucose 40 g/l | 8.6 | 14.9 |
| Glucose 20 g/l + reagent | 10.4 | 13.3 |
| glycerol 20 g/l | | |
| Reagent glycerol 40 g/l | 10.0 | 13.4 |
| Glucose 20 g/l + crude | 10.7 | 14.3 |
| glycerol GLYREX 20 g/l | | |
| Crude glycerol GLYREX 40 g/l | 9.9 | 14.5 |

### Example 4

### L-Lysine Production with Various Crude Glycerols

The Escherichia coli WC196LC/pCABD2 strain, which is an L-lysine-producing bacterium, was cultured at 37° C. for 24 hours on LB agar medium (10 g/L of tryptone, 5 g/L of yeast extract, 10 g/L of NaCl, 15 g/L of agar) containing 20 mg/L of streptomycin sulfate. The cells on the agar medium were scraped, inoculated into 20 mL of an L-lysine production medium containing 20 mg/L of streptomycin sulfate in a 500 ml-volume Sakaguchi flask, and cultured at 37° C. for 48 hours. The carbon source in the main culture was either glucose, reagent glycerol, GLYREX, Nowit DCA-F, or R Glycerin. The total amount of the carbon source was 40 g/L for all the sources.

After completion of the culture, consumption of the added saccharide and glycerol was confirmed with BF-5 (Oji Scientific Instruments), and the degree of growth was measured by determining the turbidity (OD) at 600 nm. The amount of L-lysine was measured with a Biotech Analyzer AS210 (Sakura Seiki). Averages of the results of the culture performed in flasks in duplicate are shown in Table 4.

Compared with the amount of L-lysine observed when regent glucose is the carbon source, the amount of L-lysine increased when the crude glycerols, GLYREX, Nowit DCA-F, and R Glycerin were used.

TABLE 4

| Carbon source | OD | Lys (g/l) |
|---|---|---|
| Glucose 40 g/l | 9.7 | 14.9 |
| Crude glycerol GLYREX 40 g/l | 9.8 | 16.6 |
| Crude glycerol Nowit DCA-F 40 g/l | 10.0 | 15.9 |
| Crude glycerol R Glycerin 40 g/l | 9.9 | 16.4 |

### Example 5

### Construction of L-Glutamic Acid-Producing Pantoea ananatis Strain

The plasmid RSFPPG was constructed, which essentially is the plasmid RSFCPG with the Escherichia coli citrate synthase (gltA), phosphoenolpyruvate carboxylase (ppc), and glutamate dehydrogenase (gdhA) genes (refer to European Patent Laid-open No. 1233068), and the gltA gene is replaced with the Escherichia coli methyl citrate synthase gene (prpC) (International Patent Publication WO2006/051660).

Primer 1 (SEQ ID NO: 1) and Primer 2 (SEQ ID NO: 2) were designed to amplify the part of the gltA gene of RSFCPG other than the ORF. Using these primers, and RSFCPG as a template, PCR was performed, and a fragment of about 14.9 kb was obtained. Furthermore, as for the Escherichia coli methyl citrate synthase gene (prpC), PCR was performed using Primer 3 (SEQ ID NO: 3), Primer 4 (SEQ ID NO: 4) and the Escherichia coli W3110 strain chromosomal DNA as the template, and a fragment of about 1.2 kb was obtained. Both the PCR products were treated with BglII and KpnI, and ligated, and the ligation product was used to transform the Escherichia coli JM109 strain. All the colonies that appeared were collected, and plasmids were extracted as a mixture. This plasmid mixture was used to transform the Escherichia coli ME8330 strain, which is a citrate synthase (CS) deficient strain, and the cells were applied to M9 minimal medium (12.8 g/L of Na₂HPO₄.7H₂O, 0.6 g/L of K₂HPO₄, 0.5 g/L of NaCl, 1 g/L of NH₄Cl, 2 mM MgSO₄, 0.1 mM CaCl₂) containing 50 mg/L of uracil and 5 mg/L of thiamine HCl. A plasmid was extracted from the strains that appeared, as RSFPPG. The plasmid RSFPPG was introduced into the Pantoea ananatis NP106 strain, which is an L-glutamic acid-producing bacterium, to construct an L-glutamic acid-producing bacterium, NP106/RSFPPG (this strain is called “NA1 strain”).

The NP106 strain was obtained as follows. The Pantoea ananatis AJ13601 strain exemplified above was cultured overnight at 34° C. in the LBGM9 liquid medium with shaking, and the medium was diluted so that 100 to 200 colonies emerged per plate, and were applied to an LBGM9 plate containing 12.5 mg/L of tetracycline. The colonies that appeared were replicated on an LBGM9 plate containing 12.5 mg/L of tetracycline and 25 mg/L of chloramphenicol, and a strain which was sensitive to chloramphenicol was selected. The selected strain did not have pSTVCB, and was designated G106S. Furthermore, the G106S strain was cultured overnight at 34° C. in LBGM9 liquid medium with shaking, and the medium was diluted so that 100 to 200 colonies emerged per plate, and were applied to an LBGM9 plate containing no drug. The colonies that appeared were replicated onto an LBGM9 plate containing 12.5 mg/L of tetracycline, and an LBGM9 plate containing no drug, and a strain which was sensitive to tetracycline was selected. The selected strain did not contain RSFCPG, and was designated NP106. The NP106 strain obtained as described above is the same as the AJ13601 strain which does not contain RSFCPG and pSTVCB.

### Example 6

### L-Glutamic Acid Production Culture

The Pantoea ananatis NA1 strain, which is an L-glutamic acid-producing bacterium, was cultured at 34° C. for 24 hours on LBM9 agar medium (10 g/L of tryptone, 5 g/L of yeast extract, 10 g/L of NaCl, 12.8 g/L of Na₂HPO₄.7H₂O, 0.6 g/L of K₂HPO₄, 0.5 g/L of NaCl, 1 g/L of NH₄Cl, 5 g/l of glucose, 15 g/L of agar) containing 12.5 mg/L of tetracycline hydrochloride. The cells on the agar medium were scraped, inoculated into 5 mL of an L-glutamic acid production medium containing 12.5 mg/L of tetracycline hydrochloride in a test tube, and cultured at 34° C. for 24 hours. The carbon source in the main culture was either sucrose, glucose, reagent glycerol, or crude glycerol. The total amount of the carbon source was 30 g/L for all the sources.

| | |
|---|---|
| Group A: | |
| Carbon source | 30 g/L |
| MgSO₄•7H₂O | 0.5 g/L |
| Group B: | |
| (NH₄)₂SO₄ | 20 g/L |
| KH₂PO₄ | 2 g/L |
| FeSO₄•7H₂O | 20 mg/L |
| MnSO₄•4H₂O | 20 mg/L |
| Yeast extract | 2 g/L |
| Calcium pantothenate | 18 mg/L |
| Group C: | |
| Calcium carbonate | 20 g/L |

The components of Groups A and B were subjected to autoclave sterilization at 115° C. for 10 minutes, and the component of Group C was subjected to hot air sterilization at 180° C. for 3 hours. After the components of the three groups were cooled to room temperature, they were mixed into the media.

After completion of the culture, the degree of growth was measured by determination of the turbidity (OD) at 600 nm, and consumption of the added glucose and glycerol was confirmed with BF-5 (Oji Scientific Instruments). The amounts of sucrose and L-glutamic acid were measured with a Biotech Analyzer AS 210 (Sakura Seiki). Averages of the results of the culture performed in test tubes in duplicate are shown in Table 5.

Compared with the amount of L-glutamic acid observed when glucose is the carbon source, the amount of L-glutamic acid was markedly increased when reagent glycerol was used. Furthermore, when the crude glycerol GLYREX was used, an even markedly larger L-glutamic acid amount was observed as compared to when glucose or regent glycerol was used, and a larger L-glutamic acid amount was obtained as compared to when sucrose was used.

TABLE 5

| Carbon source | OD | Glu (g/l) |
|---|---|---|
| Sucrose 40 g/l | 12.4 | 16.8 |
| Glucose 40 g/l | 13.6 | 14.1 |
| Regent glycerol 40 g/l | 14.0 | 14.9 |
| Crude glycerol GLYREX 40 g/l | 13.6 | 17.7 |

### Example 7

### Component Analysis of Crude Glycerol

Component analysis of the crude glycerols, GLYREX, Nowit DCA-F and R Glycerin, was performed. The measurement methods are as follows. Glycerol and methanol were measured by gas chromatography. Total nitrogen was measured by the Kjeldahl method, and the ether soluble fraction was measured by the Soxhlet extraction method. Formic acid and acetic acid were measured by high performance liquid chromatography, and chloride ions and sulfate ions were measured by ion chromatography. Sodium, potassium, and copper were measured by atomic absorption spectrophotometry, and phosphorus, iron, calcium, magnesium, manganese, and zinc were measured by ICP (Inductively Coupled Plasma) emission spectrometry. The results of the measurements are shown as contents per 100 g (g) in Table 6.

TABLE 6

| Measurement item | GLYREX | Nowit DCA-F | R Glycerin |
|---|---|---|---|
| Glycerol | 85.9 | 78.5 | 78.2 |
| Total nitrogen | <0.01 | <0.01 | <0.01 |
| Ether soluble fraction | 0.1 | 0.3 | <0.1 |
| Na | 0.16 | 2.09 | 2.11 |
| K | 2.45 | 0.0062 | 0.144 |
| Cl⁻ | 2.33 | 2.62 | 3.38 |
| SO₄²⁻ | <0.05 | 0.06 | <0.05 |
| Methanol | 0.0054 | 0.11 | 0.0015 |
| Formic acid | 0.02 | 0.01 | <0.01 |
| Acetic acid | 0.03 | 0.02 | 0.03 |
| P | 0.0085 | 0.0787 | 0.0219 |
| Mg | 0.001 | 0.0003 | 0.0003 |
| Fe | 0.0036 | 0.00043 | 0.00054 |
| Ca | 0.0032 | 0.0011 | <0.001 |
| Mn | 0.00003 | 0.00001 | 0.00001 |
| Cu | 0.00002 | 0.00008 | <0.00001 |
| Zn | 0.00077 | <0.00001 | <0.00001 |
| Na + K + Cl⁻ + SO₄²⁻* | 4.94 | 4.7762 | 5.63 |
| Mg + Fe + Ca* | 0.0078 | 0.00183 | 0.00084 |
| Mn + Cu + Zn* | 0.00082 | 0.00009 | 0.00001 |

*A value of 0 was used for the results below the measurement limits for calculation.

Explanation of Sequence Listing:

SEQ ID NO: 1: Primer for amplifying the part of the gltA gene other than the ORF

SEQ ID NO: 2: Primer for amplifying the part of the gltA gene other than the ORF

SEQ ID NO: 3: Primer for amplifying the prpC gene

SEQ ID NO: 4: Primer for amplifying the prpC gene

### INDUSTRIAL APPLICABILITY

According to the present invention, L-amino acids can be produced at a low cost by using a new inexpensive carbon source.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing an L-amino acid comprising culturing a bacterium belonging to the Enterobacteriaceae family which is able to produce an L-amino acid when cultured in a medium containing glycerol as the carbon source, and collecting the L-amino acid from the medium.

2. The method according to claim 1, wherein the concentration of the glycerol in the medium at the start of the culture is 1 to 30% w/v.

3. The method according to claim 1, wherein crude glycerol is added to the medium.

4. The method according to claim 3, wherein the crude glycerol is produced in biodiesel fuel production.

5. The method according to claim 3, wherein the use of crude glycerol as the carbon source in the medium results in more L-amino acid production than when the reagent glycerol is used as the carbon source.

6. The method according to claim 1, wherein the bacterium belongs to the genus Escherichia.

7. The method according to any one of claim 1, wherein the bacterium is a Pantoea bacterium.

8. The method according to claim 6, wherein the bacterium is Escherichia coli.

9. The method according to claim 1, wherein the L-amino acid is selected from the group consisting of L-threonine, L-glutamic acid, L-lysine, L-tryprophan, and combinations thereof.

10. The method according to claim 9, wherein the L-amino acid is L-threonine, and the activity of an enzyme selected from the group consisting of aspartokinase I, homoserine kinase, aspartate aminotransferase, threonine synthase which are encoded by the thr operon, aspartate semialdehyde dehydrogenase, and combinations thereof, is increased in the bacterium.

11. The method according to claim 9, wherein the L-amino acid is L-lysine, and the activity of an enzyme selected from the group consisting of dihydrodipicolinate reductase, diaminopimelate decarboxylase, diaminopimelate dehydrogenase, phosphoenolpyruvate carboxylase, aspartate aminotransferase, diaminopimelate epimerase, aspartate semialdehyde dehydrogenase, tetrahydrodipicolinate succinylase, succinyl diaminopimelate deacylase, and combinations thereof is increased, and/or the activity of lysine decarboxylase is attenuated in the bacterium.

12. The method according to claim 9, wherein the L-amino acid is L-glutamic acid, and the activity of an enzyme selected from the group consisting of glutamate dehydrogenase, citrate synthase, phosphoenolpyruvate carboxylase, methyl citrate synthase, and combinations thereof is increased, and/or activity of α-ketoglutarate dehydrogenase is attenuated in the bacterium.

13. The method according to claim 9, wherein the L-amino acid is L-tryptophan, and the activity of an enzyme selected from the group consisting of phosphoglycerate dehydrogenase, 3-deoxy-D-arabinoheptulonate-7-phosphate synthase, 3-dehydroquinate synthase, shikimate dehydratase, shikimate kinase, 5-enolpyruvate shikimate 3-phosphate synthase, chorismate synthase, prephenate dehydratase, chorismate mutase, and combinations thereof, is increased in the bacterium.
